(19) United States
(12) Patent Application Publication
(10) Pub. No.: US 2009/0082533 A1
(43) Pub. Date: Mar. 26, 2009

(54) Process for preparing low molecular weight olefin polymers, organometallic transition metal compounds biscyclopentadienyl ligand systems and catalyst systems

(75) Inventor: Michael J. Elder, Baltimore, MD (US)

Correspondence Address: Basell USA Inc., Delaware Corporate Center II, 2 Righter Parkway, Suite #300, Wilmington, DE, 19803, US

(73) Assignee: Basell Plolyolefine GmbH, Wesseling (DE)

(21) Appl. No.: 11/793,844

(22) PCT Filed: Nov. 18, 2005
(86) PCT No.: PCT/EP2005/012340

§ 371 Date: Jul. 18, 2008

Related U.S. Application Data
(60) Provisional application No. 60/638,342, filed on Dec. 22, 2004.

(30) Foreign Application Priority Data
Nov. 19, 2004 (DE) 10 2004 056 104.4

(51) Int. Cl. C07D 333/78; C08F 4/52

(52) U.S. Cl. 526/160; 549/3; 502/117

(57) ABSTRACT

The present invention relates to a process for preparing olefin polymers having a molar mass M_{w} of from 500 to 50 000 g/mol by polymerization or copolymerization of at least one olefin of the formula R^{a}—CH═CH—R^{b}, where R^{a} and R^{b} are identical or different and are each a hydrogen atom or a hydrocarbon radical having from 1 to 20 carbon atoms, or R^{a} and R^{b} together with the atoms connecting them can form a ring, at a temperature of from −60 to 200° C. and a pressure of from 0.5 to 100 bar, in solution, in suspension or in the gas phase, in the presence of hydrogen and in the presence of a catalyst system comprising at least one organometallic transition metal compound and at least one cocatalyst, wherein the organometallic transition metal compound is a compound of the formula (I), specific organometallic transition metal compounds, biscyclopentadienyl ligand systems having such a specific substitution pattern, catalyst systems comprising at least one of the organometallic transition metal compounds of the invention and the use of the biscyclopentadienyl ligand systems of the invention for preparing organometallic transition metal compounds.

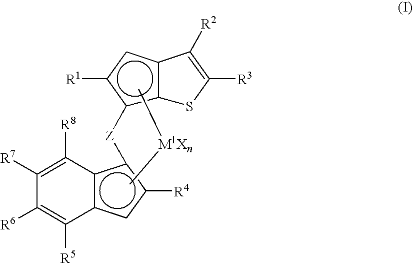

The present invention relates to a process for preparing olefin polymers having a molar mass M_{w} of from 500 to 50 000 g/mol by polymerization or copolymerization of at least one olefin of the formula R^{a}—CH═CH—R^{b}, where R^{a} and R^{b} are identical or different and are each a hydrogen atom or a hydrocarbon radical having from 1 to 20 carbon atoms, or R^{a} and R^{b} together with the atoms connecting them can form a ring, at a temperature of from −60 to 200° C. and a pressure of from 0.5 to 100 bar, in solution, in suspension or in the gas phase, in the presence of hydrogen and in the presence of a catalyst system comprising at least one organometallic transition metal compound and at least one cocatalyst, wherein the organometallic transition metal compound is a compound of the formula (I),

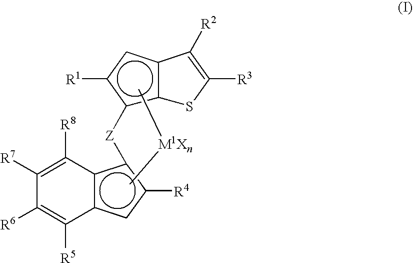

where
M¹ is an element of group 3, 4, 5 or 6 of the Periodic Table of the Elements or the lanthanides,
the radicals X are identical or different and are each an organic or inorganic radical, with two radicals X also being able to be joined to one another to form a divalent radical,
n is a natural number from 1 to 4,
R¹ is hydrogen, an organic radical which has from 1 to 40 carbon atoms and is unbranched in the α position or an organic radical which has from 1 to 40 carbon atoms and is bound via an sp²-hybridized carbon atom,
R² is an organic radical having from 1 to 40 carbon atoms,
R³ is an organic radical having from 1 to 40 carbon atoms,
R⁴ is hydrogen, an organic radical which has from 1 to 40 carbon atoms and is unbranched in the α position or an organic radical which has from 1 to 40 carbon atoms and is bound via an sp²-hybridized carbon atom,
R⁵, R⁶, R⁷, R⁸ are identical or different and are each hydrogen, halogen or an organic radical having from 1 to 40 carbon atoms or two adjacent radicals R⁵, R⁶, R⁷ or R⁸ together with the atoms connecting them form a monocyclic or polycyclic, substituted or unsubstituted ring system which has from 1 to 40 carbon atoms and may also contain heteroatoms selected from the group consisting of the elements Si, Ge, N, P, O, S, Se and Te,
   and
Z is a bridge consisting of a divalent atom or a divalent group.

The present invention further relates to specific organometallic transition metal compounds, biscyclopentadienyl ligand systems having such a specific substitution pattern, catalyst systems comprising at least one of the organometallic transition metal compounds of the invention and the use of the biscyclopentadienyl ligand systems of the invention for preparing organometallic transition metal compounds.

Research and development on the use of organometallic transition metal compounds, in particular metallocenes, as catalyst components for the polymerization and copolymerization of olefins with the objective of preparing tailored polyolefins has been pursued intensively in universities and in industry over the past 15 years.

Not only the ethene-based polyolefins prepared by means of metallocene catalyst systems but also, in particular, the propene-polyolefins prepared by means of metallocene catalyst systems now represent a dynamically growing market segment.

However, metallocene catalysts make it possible to prepare not only relatively high molecular weight polyolefins which can be processed to produce shaped bodies such as films, fibers, injection-molded parts or blow-molded bodies but also polyolefin waxes which are used, for example, as auxiliaries in plastic processing, as constituents of shoe polishes or as components in printing inks.

EP 0 571 882 describes a process for preparing a polyolefin wax by polymerization or copolymerization of olefins or diolefins in a suspension process in which various bridged or unbridged metallocenes can be used. To obtain the desired molar masses, the olefin polymerization is carried out in the presence of hydrogen as molar mass regulator. If the hydrogen concentration required for regulation becomes too great, this frequently results in a reduction in the activity of the catalyst system. Catalyst systems which have a high response to hydrogen and a high activity are therefore sought.

Owing to the solubility of hydrogen in organic liquids, setting of high hydrogen concentrations in solution or suspension processes present additional technical problems compared to gas-phase processes.

To prepare particularly high-melting, i.e. highly isotactic, polyolefin waxes, metallocene catalysts based on metallocenes as described in U.S. Pat. No. 5,455,366 or U.S. Pat. No. 6,444,833 are possibilities. However, the metallocene catalysts described there experimentally require excessively high hydrogen concentrations to achieve a sufficient reduction in the molar masses in the olefin polymerization.

It was therefore an object of the present invention to find a process for preparing polyolefin waxes in the presence of organometallic transition metal compounds which as catalyst constituent are able to produce polyolefin waxes at high activity and reduce hydrogen concentrations compared to the metallocenes used hitherto.

We have accordingly found the process mentioned at the outset and defined in the claims for preparing olefin polymers having a molar mass M_{w} of from 500 to 50 000 g/mol, specific organometallic transition metal compounds, biscyclopentadienyl ligand systems, catalyst systems comprising at least one of the organometallic transition metal compounds according to the invention and the use of the biscyclopentadienyl ligand systems of the invention for preparing organometallic transition metal compounds.

The process of the invention is preferably used for preparing olefin polymers having a molar mass M_{w} of from 1000 to 30 000 g/mol, in particular from 2000 to 20 000 g/mol.

In the process of the invention, at least one olefin of the formula R^{a}—CH═CH—R^{b}, where R^{a} and R^{b} are identical or different and are each a hydrogen atom or a hydrocarbon radical having from 1 to 20 carbon atoms, in particular from 1 to 10 carbon atoms, or R^{a} and R^{b} together with the atoms connecting them can form one or more rings, is polymerized.

Examples of such olefins are 1-olefins having from 2 to 20, preferably from 2 to 10, carbon atoms, e.g. ethene, propene, 1-butene, 1-pentene, 1-hexene, 1-heptene, 1-octene, 1-decene or 4-methyl-1-pentene, or unsubstituted or substituted vinylaromatic compounds such as styrene and styrene derivatives, or dienes such as 1,3-butadiene, 1,4-hexadiene, 1,7-octadiene, 5-ethylidene-2-norbornene, norbornadiene, ethylnorbornadiene or cyclic olefins such as norbornene, tetracyclododecene or methylnorbornene.

In the process of the invention, preference is given to homopolymerizing propene or copolymerizing propene with ethene, 1-butene, 1-pentene, 1-hexene and/or 1-octene and/or cyclic olefins such as norbornene and/or dienes having from 4 to 20 carbon atoms, e.g. 1,4-hexadiene, norbornadiene, ethylidenenorbornene or ethylnorbornadiene, in particular copolymerizing propene with ethene. The process of the invention is very particularly suitable for the preparation of polypropylene waxes.

The process of the invention can be carried out at temperatures in the range from −60 to 300° C., preferably in the range from 0 to 150° C., in particular from 50 to 100° C.

The process of the invention can be carried out at a pressure of from 0.5 to 100 bar, preferably from 5 to 65 bar.

The process of the invention can be carried out in a known manner in solution, in suspension or in the gas phase in the customary reactors used for the polymerization of olefins. The process is preferably carried out in solution or suspension. The process can be carried out batchwise or preferably continuously in one or more stages. As solvents or suspension media, it is possible to use inert hydrocarbons, for example propane, isobutane or hexane, or else the monomers themselves, for example propene.

The mean residence time in the process of the invention is usually from 0.5 to 5 hours, preferably from 0.5 to 3 hours.

As molar mass regulator, hydrogen is used in the process of the invention. Since the amount of hydrogen necessary for regulating the molar mass depends on the polymerization process, a person skilled in the art will determine the amount of hydrogen required to obtain a desired molecular weight of the olefin polymer in the process employed by means of a few routine tests.

The process of the invention is carried out in the presence of a catalyst system which comprises at least one organometallic transition metal compound and at least one cocatalyst and in which the transition metal compound is a compound of the formula (I) as described at the outset.

M¹ is an element of group 3, 4, 5 or 6 of the Periodic Table of the Elements or the lanthanides, for example titanium, zirconium, hafnium, vanadium, niobium, tantalum, chromium, molybdenum or tungsten, preferably titanium, zirconium, hafnium, particularly preferably zirconium or hafnium, and very particularly preferably zirconium.

The radicals X are identical or different, preferably identical, and are each an organic or inorganic radical, with two radicals X also being able to be joined to one another. X is preferably halogen, for example fluorine, chlorine, bromine, iodine, preferably chlorine, hydrogen, C₁-C₂₀-, preferably C₁-C₄-alkyl, in particular methyl, C₂-C₂₀-, preferably C₂-C₄-alkenyl, C₆-C₂₂-, preferably C₆-C₁₀-aryl, an alkylaryl or arylalkyl group having from 1 to 10, preferably from 1 to 4, carbon atoms in the alkyl radical and from 6 to 22, preferably from 6 to 10, carbon atoms in the aryl radical, —OR⁹ or —NR⁹R¹⁰, preferably —OR⁹, with two radicals X also being able to be joined to one another, preferably two radicals —OR⁹. Furthermore, the two radicals X can form a substituted or unsubstituted diene ligand, in particular a 1,3-diene ligand. The radicals R⁹ and R¹⁰ are each C₁-C₁₀-, preferably C₁-C₄-alkyl, C₆-C₁₅-, preferably C₆-C₁₀-aryl, alkylaryl, arylalkyl, fluoroalkyl or fluoroaryl each having from 1 to 10, preferably from 1 to 4, carbon atoms in the alkyl radical and from 6 to 22, preferably from 6 to 10, carbon atoms in the aryl radical.

Unless restricted further, alkyl is a linear, branched or cyclic radical such as methyl, ethyl, n-propyl, isopropyl, n-butyl, i-butyl, s-butyl, t-butyl, n-pentyl, cyclopentyl, n-hexyl, cyclohexyl, n-heptyl or n-octyl.

The index n is a natural number from 1 to 4 which is frequently equal to the oxidation number of M¹ minus 2. In the case of the element of group 4 of the Periodic Table of the Elements, n is preferably 2.

The radical R¹ is hydrogen, an organic radical which has from 1 to 40 carbon atoms and is unbranched in the α position or an organic radical which has from 1 to 40 carbon atoms and is bound via an sp²-hybridized carbon atom, with an organic radical which has from 1 to 40 carbon atoms and is unbranched in the α position being a radical of this type whose linking a atom is bound to not more than one atom different from hydrogen. The linking a atom of the radical which has from 1 to 40 carbon atoms and is unbranched in the α position is preferably a carbon atom. Preferred examples of an organic radical bound via an sp²-hybridized carbon atom are substituted or unsubstituted C₆-C₂₀-aryl radicals or substituted or unsubstituted, heteroaromatic radicals which have from 2 to 40, in particular from 3 to 30, carbon atoms and contain at least one heteroatom, preferably selected from the group consisting of the elements O, N, S and P, in particular O, N and S.

The radical R¹ is particularly preferably a C₁-C₁₀-n-alkyl radical such as methyl, ethyl, n-propyl, n-butyl, n-pentyl or n-hexyl, in particular methyl or ethyl.

The radical R² is an organic radical having from 1 to 40 carbon atoms, for example C₁-C₄₀-alkyl, C₁-C₁₀-fluoroalkyl, C₂-C₄₀-alkenyl, C₆-C₄₀-aryl, C₆-C₁₀-fluoroaryl, arylalkyl, arylalkenyl or alkylaryl having from 1 to 10, preferably from 1 to 4, carbon atoms in the alkyl radical and from 6 to 22, preferably from 6 to 10, carbon atoms in the aryl radical, a saturated heterocycle having from 2 to 40 carbon atoms or a C₂-C₄₀-heteroaromatic radical having in each case at least one heteroatom selected from the group consisting of the elements O, N, S, P and Se, in particular O, N and S, with the heteroaromatic radical being able to be substituted by further radicals R¹¹, where R¹¹ is an organic radical having from 1 to 20 carbon atoms, for example C₁-C₁₀-, preferably C₁-C₄-alkyl, C₈-C₁₅-, preferably C₆-C₁₀-aryl, alkylaryl, arylalkyl, fluoroalkyl or fluoroaryl each having from 1 to 10, preferably from 1 to 4, carbon atoms in the alkyl radical and from 6 to 18, preferably from 6 to 10, carbon atoms in the aryl radical, and a plurality of radicals R¹¹ can be identical or different. R² is preferably a substituted or unsubstituted C₆-C₄α-aryl radical or C₂-C₄₀-heteroaromatic radical having at least one heteroatom selected from the group consisting of the elements O, N, S and P. The radical R² is particularly preferably a substituted or unsubstituted C₆-C₄₀-aryl or alkylaryl radical having from 1 to 10, preferably from 1 to 4, carbon atoms in the alkyl radical and from 6 to 22, preferably from 6 to 10, carbon atoms in the aryl radical, with the radicals also being able to be halogenated. Examples of particularly preferred radicals R² are phenyl, 2-tolyl, 3-tolyl, 4-tolyl, 2,3-dimethylphenyl, 2,4-dimethylphenyl, 2,5-dimethylphenyl, 2,6-dimethylphenyl, 3,4-dimethylphenyl, 3,5-dimethylphenyl, 3,5-di(tert-butyl)phenyl, 2,4,6-trimethylphenyl, 2,3,4-trimethylphenyl, 1-naphthyl, 2-naphthyl, phenanthrenyl, p-isopropylphenyl, p-tert-butylphenyl, p-s-butylphenyl, p-cyclohexylphenyl and p-trimethylsilylphenyl, in particular phenyl, 1-naphthyl, 3,5-dimethylphenyl and p-tert-butylphenyl.

The radical R³ is an organic radical having from 1 to 40 carbon atoms, for example C₁-C₄₀-alkyl, C₁-C₁₀-fluoroalkyl, C₂-C₄₀-alkenyl, C₆-C₄₀-aryl, C₆-C₁₀-fluoroaryl, arylalkyl, arylalkenyl or alkylaryl having from 1 to 10, preferably from 1 to 4, carbon atoms in the alkyl radical and from 6 to 22, preferably from 6 to 10, carbon atoms in the aryl radical, a saturated heterocycle having from 2 to 40 carbon atoms or a C₂-C₄₀-heteroaromatic radical having in each case at least one heteroatom selected from the group consisting of the elements O, N, S, P and Se, in particular O, N and S, with the heteroaromatic radical being able to be substituted by further radicals R¹¹, where R¹¹ is an organic radical having from 1 to 20 carbon atoms, for example C₁-C₁₀-, preferably C₁-C₄-alkyl, C₆-C₁₅-, preferably C₆-C₁₀-aryl, alkylaryl, arylalkyl, fluoroalkyl or fluoroaryl each having from 1 to 10, preferably from 1 to 4, carbon atoms in the alkyl radical and from 6 to 18, preferably from 6 to 10, carbon atoms in the aryl radical, and a plurality of radicals R¹¹ can be identical or different. R³ is preferably a C₁-C₁₀-n-alkyl radical or a substituted or unsubstituted C₆-C₄₀-aryl radical or C₂-C₄₀-heteroaromatic radical having at least one heteroatom selected from the group consisting of the elements O, N, S and P. The radical R³ is particularly preferably a C₁-C₁₀-n-alkyl radical such as methyl, ethyl, n-propyl, n-butyl, n-pentyl or n-hexyl, in particular methyl or ethyl.

The radical R⁴ is hydrogen, an organic radical which has from 1 to 40 carbon atoms and is unbranched in the α position or an organic radical which has from 1 to 40 carbon atoms and is bound via an sp²-hybridized carbon atom, with an organic radical which has from 1 to 40 carbon atoms and is unbranched in the α position being a radical of this type whose linking a atom is bound to not more than one atom different from hydrogen. The linking a atom of the radical which has from 1 to 40 carbon atoms and is unbranched in the α position is preferably a carbon atom. Preferred examples of an organic radical bound via an sp²-hybridized carbon atom are substituted or unsubstituted C₆-C₂₀-aryl radicals or substituted or unsubstituted, heteroaromatic radicals which have from 2 to 40, in particular from 3 to 30, carbon atoms and contain at least one heteroatom, preferably selected from the group consisting of the elements O, N, S and P, in particular O, N and S.

The radical R⁴ is particularly preferably a C₁-C₁₀-n-alkyl radical such as methyl, ethyl, n-propyl, n-butyl, n-pentyl or n-hexyl, in particular methyl or ethyl.

The radicals R⁵, R⁶, R⁷ and R⁸ are identical or different and are each hydrogen, halogen such as fluorine, chlorine, bromine or iodine, preferably fluorine, or an organic radical having from 1 to 40 carbon atoms, for example a cyclic, branched or unbranched C₁-C₂₀-, preferably C₁-C₈-alkyl radical, a C₂-C₂₀-, preferably C₂-C₈-alkenyl radical, a C₆-C₂₂-, preferably C₆-C₁₀-aryl radical, an alkylaryl or arylalkyl radical having from 1 to 10, preferably from 1 to 4, carbon atoms in the alkyl radical and from 6 to 22, preferably from 6 to 10, carbon atoms in the aryl radical, with the radicals also being able to be halogenated, unsaturated heterocycles having from 2 to 40 carbon atoms or a C₂-C₄₀-heteroaromatic radical having at least one heteroatom selected from the group consisting of the elements O, N, S, P and Se, in particular O, N and S, with the heteroaromatic radical being able to be substituted by further radicals R¹¹, where R¹¹ is an organic radical having from 1 to 20 carbon atoms, for example C₁-C₁₀, preferably C₁-C₄-alkyl, C₆-C₁₅-, preferably C₆-C₁₀-aryl, alkylaryl, arylalkyl, fluoroalkyl or fluoroaryl each having from 1 to 10, preferably from 1 to 4, carbon atoms in the alkyl radical and from 6 to 18, preferably from 6 to 10, carbon atoms in the aryl radical, and a plurality of radicals R¹¹ can be identical or different, or two adjacent radicals R⁵, R⁶, R⁷ and R⁸ together with the atoms connecting them form a monocyclic or polycyclic, substituted or unsubstituted ring system which has from 4 to 40 carbon atoms and may also contain heteroatoms selected from the group consisting of the elements Si, Ge, N, P, O, S, Se and Te, in particular N and S.

R⁵ and R⁶ preferably together form a substituted or unsubstituted, in particular unsubstituted, 1,3-butadiene-1,4-diyl group, and R⁷ and R⁸ are preferably each hydrogen.

Z is a bridge consisting of a divalent atom or a divalent group. Examples of Z are:

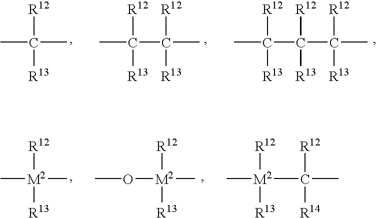

—B(R¹²)—, —B(NR¹²R¹³)—, —Al(R¹²)—, —O—, —S—, —S(O)—, —S((O)₂)—, —N(R¹²)—, —C(O)—, —P(R¹²)— or —P(O)(R¹²)—,
   in particular

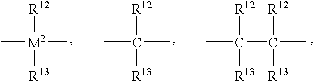

where
M² is silicon, germanium or tin, preferably silicon or germanium, particularly preferably silicon, and
R¹², R¹³ and R¹⁴ are identical or different and are each a hydrogen atom, a halogen atom, a trimethylsilyl group, a C₁-C₁₀-, preferably C₁-C₃-alkyl group, a C₁-C₁₀-fluoroalkyl group, a C₆-C₁₀-fluoroaryl group, a C₆-C₁₀-aryl group, a C₁-C₁₀-, preferably C₁-C₃-alkoxy group, a C₇-C₁₅-alkylaryloxy group, a C₂-C₁₀-, preferably C₂-C₄-alkenyl group, a C₇-C₄₀-arylalkyl group, a C₈-C₄₀-arylalkenyl group or a C₇-C₄₀-alkylaryl group, or two adjacent radicals together with the atoms connecting them form a saturated or unsaturated ring having from 4 to 15 carbon atoms.

Preferred embodiments of Z are the bridges:

dimethylsilanediyl, methylphenylsilanediyl, diphenylsilanediyl, methyl-tert-butylsilanediyl, dimethylgermanediyl, ethylidene, 1-methylethylidene, 1,1-dimethylethylidene, 1,2-dimethylethylidene, 1,1,2,2-tetramethylethylidene, dimethylmethylidene, phenylmethylmethylidene or diphenylmethylidene, in particular dimethylsilanediyl, diphenylsilanediyl and ethylidene.

Z is particularly preferably a substituted silylene group or a substituted or unsubstituted ethylene group, preferably a substituted silylene group such as dimethylsilanediyl, methylphenylsilanediyl, methyl-tert-butylsilanediyl or diphenylsilanediyl, in particular dimethylsilanediyl.

According to the invention, the radicals R¹, R², R³, R⁴, R⁵, R⁶, R⁷, R⁸, R⁹, R¹⁰, R¹¹, R¹², R¹³ and R¹⁴ can contain further heteroatoms, in particular selected from the group consisting of the elements Si, N, P, O, S, F and Cl, or functional groups in place of hydrocarbon radicals, carbon atoms or hydrogen atoms without altering the polymerization properties of the organometallic transition metal compound using the process of the invention, as long as these heteroatoms or functional groups are chemically inert under the polymerization conditions.

Furthermore, the substituents are, unless restricted further, defined as follows for the purposes of the present invention:

The term “organic radical having from 1 to 40 carbon atoms” as used in the present text refers, for example, to C₁-C₄₀-alkyl radicals, C₁-C₁₀-fluoroalkyl radicals, C₁-C₁₂-alkoxy radicals, saturated C₃-C₂₀-heterocyclic radicals, C₆-C₄₀-aryl radicals, C₂-C₄₀-heteroaromatic radicals, C₆-C₁₀-fluoroaryl radicals, C₆-C₁₀-aryloxy radicals, silyl radicals having from 3 to 24 carbon atoms, C₂-C₂₀-alkenyl radicals, C₂-C₂₀-alkynyl radicals, C₇-C₄₀-arylalkyl radicals or C₈-C₄₀-arylalkenyl radicals. Such an organic radical is in each case derived from an organic compound. Thus, the organic compound methanol can in principle give rise to three different organic radicals each having one carbon atom, namely methyl (H₃C—), methoxy (H₃C—O—) and hydroxymethyl (HOC(H₂)—).

The term “alkyl” as used in the present text encompasses linear or singly or multiply branched saturated hydrocarbons which may also be cyclic. Preference is given to a C₁-C₁₈-alkyl radical such as methyl, ethyl, n-propyl, n-butyl, n-pentyl, n-hexyl, n-heptyl, n-octyl, n-nonyl, n-decyl, cyclopentyl, cyclohexyl, isopropyl, isobutyl, isopentyl, isohexyl, sec-butyl or tert-butyl.

The term “alkenyl” as used in the present text encompasses linear or singly or multiply branched hydrocarbons having one or more C—C double bonds which may be cumulated or alternating.

The term “saturated heterocyclic radical” as used in the present text refers, for example, to monocyclic or polycyclic, substituted or unsubstituted hydrocarbon radicals in which one or more carbon atoms, CH groups and/or CH₂ groups are replaced by heteroatoms, preferably selected from the group consisting of the elements O, S, N and P. Preferred examples of substituted or unsubstituted saturated heterocyclic radicals are pyrrolidinyl, imidazolidinyl, pyrazolidinyl, piperidyl, piperazinyl, morpholinyl, tetrahydrofuranyl, tetrahydropyranyl, tetrahydrothiophenyl and the like, and also methyl-, ethyl-, propyl-, isopropyl- and tert-butyl-substituted derivatives thereof.

The term “aryl” as used in the present text refers, for example, to aromatic and optionally fused polyaromatic hydrocarbon radicals which may be monosubstituted or polysubstituted by linear or branched C₁-C₁₈-alkyl, C₁-C₁₈-alkoxy, C₂-C₁₀-alkenyl or halogen, in particular fluorine. Preferred examples of substituted and unsubstituted aryl radicals are, in particular, phenyl, pentafluorophenyl, 4-methylphenyl, 4-ethylphenyl, 4-n-propylphenyl, 4-isopropylphenyl, 4-tert-butylphenyl, 4-methoxyphenyl, 1-naphthyl, 9-anthryl, 9-phenanthryl, 3,5-dimethylphenyl, 3,5-di-tert-butylphenyl or 4-trifluoromethylphenyl.

The term “heteroaromatic radical” as used in the present text refers, for example, to aromatic hydrocarbon radicals in which one or more carbon atoms have been replaced by nitrogen, phosphorus, oxygen or sulfur atoms or combinations thereof. These may, like the aryl radicals, be monosubstituted or polysubstituted by linear or branched C₁-C₁₈-alkyl, C₂-C₁₀-alkenyl or halogen, in particular fluorine. Preferred examples are furyl, thienyl, pyrrolyl, pyridyl, pyrazolyl, imidazolyl, oxazolyl, thiazolyl, pyrimidinyl, pyrazinyl and the like, and also methyl-, ethyl, propyl-, isopropyl- and tert-butyl-substituted derivatives thereof.

The term “arylalkyl” as used in the present text refers, for example, to aryl-containing substituents whose aryl radical is bound via an alkyl chain to the remainder of the molecule. Preferred examples are benzyl, substituted benzyl, phenethyl, substituted phenethyl and the like.

The terms fluoroalkyl and fluoroaryl mean that at least one hydrogen atom, preferably a plurality of and at most all, hydrogen atoms of the corresponding radical have been replaced by fluorine atoms. Examples of fluorine-containing radicals which are preferred according to the invention are trifluoromethyl, 2,2,2-trifluoroethyl, pentafluorophenyl, 4-trifluoromethylphenyl, 4-perfluoro-tert-butylphenyl and the like.

In the process of the invention, preference is given to using organometallic transition metal compounds of the formula (I) in which
R¹ is a C₁-C₁₀-n-alkyl radical, preferably a C₁-C₆-n-alkyl radical such as methyl, ethyl, n-propyl, n-butyl, n-pentyl or n-hexyl, in particular methyl or ethyl,
R² is a substituted or unsubstituted C₆-C₄₀-aryl radical or C₂-C₄₀-heteroaromatic radical having at least one heteroatom selected from the group consisting of the elements O, N, S and P, preferably a substituted or unsubstituted C₆-C₄₀-aryl or alkylaryl radical having from 1 to 10, preferably from 1 to 4, carbon atoms in the alkyl radical and from 6 to 22, preferably from 6 to 10, carbon atoms in the aryl radical, with the radicals also being able to be halogenated, for example phenyl, 2-tolyl, 3-tolyl, 4-tolyl, 2,3-dimethylphenyl, 2,4-dimethylphenyl, 2,5-dimethylphenyl, 2,6-dimethylphenyl, 3,4-dimethylphenyl, 3,5-dimethylphenyl, 3,5-di(tert-butyl)phenyl, 2,4,6-trimethylphenyl, 2,3,4-trimethylphenyl, 1-naphthyl, 2-naphthyl, phenanthrenyl, p-isopropylphenyl, p-tert-butylphenyl, p-s-butylphenyl, p-cyclohexylphenyl and p-trimethylsilylphenyl, in particular phenyl, 1-naphthyl, 3,5-dimethylphenyl and p-tert-butylphenyl,
R³ is a C₁-C₁₀-n-alkyl radical or substituted or unsubstituted C₆-C₄₀-aryl radical or C₂-C₄₀-heteroaromatic radical having at least one heteroatom selected from the group consisting of the elements O, N, S and P, preferably a C₁-C₁₀-n-alkyl radical such as methyl, ethyl, n-propyl, n-butyl, n-pentyl or n-hexyl, in particular methyl or ethyl,
   and
the other variables and indices are as defined for the formula (I).

In the process of the invention, particular preference is given to using organometallic transition metal compounds of the formula (I) as defined above

in which
R⁴ is a C₁-C₁₀-n-alkyl radical, preferably a C₁-C₆-n-alkyl radical such as methyl, ethyl, n-propyl, n-butyl, n-pentyl or n-hexyl, in particular methyl or ethyl,
R⁵, R⁶ together form a substituted or unsubstituted, in particular unsubstituted 1,3-butadiene-1,4-diyl group,
R⁷, R⁸ are each hydrogen,
   and
the other variables and indices are as defined from the formula (I).

The invention further provides the organometallic transition metal compounds of the formula (Ia) described in the claims

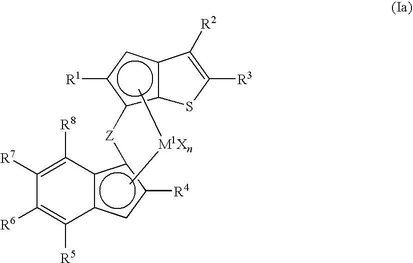

where
R⁵, R⁶ together form a substituted or unsubstituted, in particular unsubstituted, 1,3-butadiene-1,4-diyl group,
R⁷, R⁸ are identical or different and are each hydrogen, halogen or an organic radical having from 1 to 40 carbon atoms, in particular hydrogen,
   and
the variables M¹, X, n, R¹, R², R³, R⁴ and Z are as defined for the formula (I).

Illustrative but nonlimiting examples of transition metal compounds of the formula (I) or (Ia) which can be used in the process of the invention are:
Me₂Si(2,5-Me₂-3-Ph-cyclopenta[2,3-b]thiophen-6-yl)(2-Me-4,5-benzoinden-1-yl)ZrCl₂,
Me₂Si(2,5-Me₂-3-Ph-cyclopenta[2,3-b]thiophen-6-yl)(2-iPr-4,5-benzoinden-1-yl)ZrCl₂,
Me₂Si(2,5-Me₂-3-Ph-cyclopenta[2,3-b]thiophen-6-yl)(2-(5-Me-furan-2-yl)-4,5-benzoinden-1-yl)ZrCl₂,
Me₂Si(2,5-Me₂-3-Ph-cyclopenta[2,3-b]thiophen-6-yl)(2-Me-inden-1-yl)ZrCl₂,
Me₂Si(2,5-Me₂-3-Ph-cyclopenta[2,3-b]thiophen-6-yl)(2-Me-4-(4-tBu-Ph)-inden-1-yl)ZrCl₂,
Me₂Si(2-Ph-3,5-Me₂-cyclopenta[2,3-b]thiophen-6-yl)(2-Me-4,5-benzoinden-1-yl)ZrCl₂,
Me₂Si(2-Me-3-Ph-5-iPr-cyclopenta[2,3-b]thiophen-6-yl)(2-Me-4,5-benzoinden-1-yl)ZrCl₂,
Me₂Si(2,5-Me₂-3-Ph-cyclopenta[2,3-b]thiophen-6-yl)(2-Me-4-Ph-6-Me-inden-1-yl)ZrCl₂,
Me₂Si(2,5-Me₂-3-Ph-cyclopenta[2,3-b]thiophen-6-yl)(2-Me-4,6-iPr₂-inden-1-yl)ZrCl₂,
Me₂Si(2,5-Me₂-3-Ph-cyclopenta[2,3-b]thiophen-6-yl)(2-Me-4,5-benzoinden-1-yl)HfCl₂.

In the process of the invention and when using the novel organometallic transition metal compounds of the formula (Ia), less hydrogen is required to reduce the molar mass of the polyolefins, in particular polypropylenes, so as to produce polyolefin waxes compared to the processes and metallocenes known hitherto. Furthermore, the process of the invention and the novel organometallic transition metal compounds of the formula (Ia) display an increased activity compared to the prior art in the polymerization of olefins, in particular propylene.

The novel metallocenes of the formula (Ia) and the metallocenes of the formula (I) used in the process of the invention can be prepared by methods as described in WO 01/48034. In these methods, the organometallic transition metal compounds of the formula (I) or (Ia) are usually obtained together with a further diastereomer.

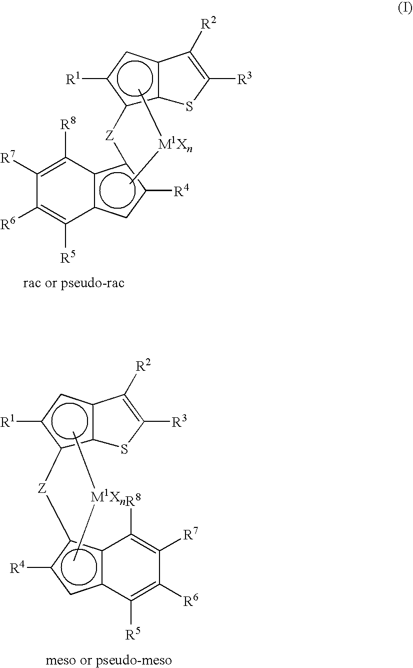

The organometallic transition metal compounds of the formula (I) or (Ia) (rac or pseudo-rac) can be used as a diastereomer mixture together with the undesired diastereomers (meso or pseudomeso) produced in their synthesis in the production of catalysts. The organometallic transition metal compounds of the formula (I) or (Ia) give highly isotactic polypropylene, while the corresponding undesired diastereomers generally give atactic polypropylene.

The separation of the diastereomers is known in principle.

The invention further provides biscyclopentadienyl ligand systems of the formula (II)

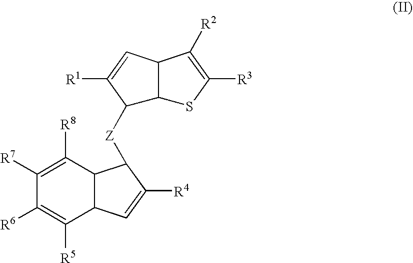

or its double bond isomers,
where the variables R¹, R², R³, R⁴, R⁵, R⁶, R⁷, R⁸ and Z are as defined for the formula (Ia).

The substitution pattern of the biscyclopentadienyl ligand systems of the formula (II) is critical for the particular polymerization properties of the organometallic transition metal compounds in which these biscyclopentadienyl ligand systems are present.

The invention further provides for the use of a biscyclopentadienyl ligand system of the formula (II) for preparing an organometallic transition metal compound, preferably for preparing an organometallic transition metal compound of an element of group 4 of the Periodic Table of the Elements, in particular zirconium.

Thus, the present invention also provides a process for preparing an organometallic transition metal compounds, which comprises reacting a biscyclopentadienyl ligand system of the formula (II) or a bisanion prepared there from with a transition metal compound. A ligand system of the formula (II) is usually firstly doubly deprotonated by means of a strong base, for example n-butyllithium, and subsequently reacted with a suitable transition metal source, for example zirconium tetrachloride. However, an alternative is to react the uncharged biscyclopentadienyl ligand system of the formula (II) directly with a suitable transition metal source which has strongly basic ligands, for example, tetrakis(dimethylamino)zirconium.

The catalyst system used in the process of the invention comprises not only at least one of the abovementioned organometallic transition metal compounds of the formula (I) or (Ia) but also at least one cocatalyst.

The cocatalyst, which together with the organometallic transition metal compounds of the formula (I) or (Ia) forms a polymerization-active catalyst system, is able to convert the organometallic transition metal compound into a species which is polymerization-active toward at least one olefin. The cocatalyst is therefore sometimes also referred to activating compound. The polymerization active transition metal species is frequently a cationic species. In this case, the cocatalyst is frequently also referred to as cation-forming compound.

The present invention further provides a catalyst system for the polymerization of olefins, in particular for the preparation of polypropylene wax, which comprises at least one organometallic transition metal compound of the formula (Ia) and at least one cocatalyst which is able to convert the organometallic transition metal compound into a species which is polymerization-active toward at least one olefin.

Suitable cocatalysts or cation-forming compounds are, for example, compounds such as an aluminoxane, a strong uncharged Lewis acid, an ionic compound having a Lewis-acid cation or an ionic compound containing a Brönsted acid as cation. Preference is given to using an aluminoxane as cocatalyst in the process of the invention or together with the novel organometallic transition metal compound of the formula (Ia).

Particular preference is given to a process according to the invention for preparing olefin polymers in which the organometallic transition metal compound of the formula (I) or (Ia) is preactivated with an aluminoxane prior to use in the polymerization reaction. In this preactivation step, the organometallic transition metal compound, for example as such or in solution, is brought into contact with an aluminoxane, in particular a solution of a methylaluminoxane, for some time, e.g. for from 1 minute to 48 hours, preferably from 5 minutes to 4 hours, in order to form the catalyst system.

In the case of metallocene complexes as organometallic transition metal compound, the cocatalysts are frequently also referred to as compounds capable of forming metallocenium ions.

As aluminoxanes, it is possible to use, for example, the compounds described in WO 00/31090. Particularly useful aluminoxanes are open-chain or cyclic aluminoxane compounds of the general formula (XI) or (XII)

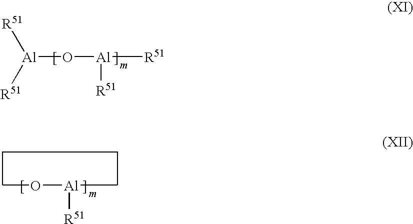

where
R⁵¹ is a C₁-C₄-alkyl group, preferably a methyl or ethyl group, and m is an integer from 5 to 30, preferably from 10 to 25.

These oligomeric aluminoxane compounds are usually prepared by reacting a solution of trialkylaluminum with water. In general, the oligomeric aluminoxane compounds obtained in this way are in the form of mixtures of both linear and cyclic chain molecules of various lengths, so that m is to be regarded as a mean. The aluminoxane compounds can also be used in admixture with other metal alkyls, preferably aluminum alkyl.

Furthermore, modified aluminoxanes in which some of the hydrocarbon radicals or hydrogen atoms have been replaced by alkoxy, aryloxy, siloxy or amide radicals can also be used in place of the aluminoxane compounds of the general formula (XI) or (XII).

It has been found to be advantageous to use the organometallic transition metal compound of the formula (I) or (Ia) and the aluminoxane compounds in the process of the invention in such amounts that the atomic ratio of aluminum from the aluminoxane compounds to the transition metal from the organometallic transition metal compound is in the range from 1:1 to 100 000:1, preferably in the range from 5:1 to 20 000:1 and in particular in the range from 10:1 to 2000:1.

As strong, uncharged, Lewis acids, preference is given to compounds of the general formula (XIII)

M³X¹X²X³ (XIII)

where
M³ is an element of group 13 of the Periodic Table of the Elements, in particular B, Al or Ga, preferably B,
X¹, X² and X³ are each, independently of one another, hydrogen, C₁-C₁₀-alkyl, C₆-C₁₅-aryl, alkylaryl, arylalkyl, haloalkyl or haloaryl each having from 1 to 10 carbon atoms in the alkyl radical and from 6 to 20 carbon atoms in the aryl radical or fluorine, chlorine, bromine or iodine, in particular haloaryl, preferably pentafluorophenyl.

Further examples of strong, uncharged Lewis acids are given in WO 00/31090.

Particular preference is given to compounds of the general formula (XIII) in which X¹, X² and X³ are identical, preferably tris(pentafluorophenyl)borane.

Strong uncharged Lewis acids suitable as cocatalyst or cation-forming compounds also include the reaction products of a boronic acid with two equivalents of an aluminum trialkyl or the reaction products of an aluminum trialkyl with two equivalents of an acidic fluorinated, in particular perfluorinated, hydrocarbon compound such as pentafluorophenol or bis(pentafluorophenyl)borinic acid.

Suitable ionic compounds having Lewis acid cations include salt-like compounds of the cation of the general formula (XIV)

[(Y^{a+})Q¹Q² . . . Q^{z}]^{d+} (XIV)

where
Y is an element of groups 1 to 16 of the Periodic Table of the Elements,
Q¹ to Q^{z} are singly negatively charged groups such as C₁-C₂₈-alkyl, C₆-C₁₅-aryl, alkylaryl, arylalkyl, haloalkyl, haloaryl each having from 6 to 20 carbon atoms in the aryl radical and from 1 to 28 carbon atoms in the alkyl radical, C₃-C₁₀-cycloalkyl which may bear C₁-C₁₀-alkyl groups as substituents, halogen, C₁-C₂₈-alkoxy, C₆-C₁₅-aryloxy, silyl or mercaptyl groups,
a is an integer from 1 to 6 and
z is an integer from 0 to 5, and
d corresponds to the difference a−z, but d is greater than or equal to 1.

Particularly useful cations are carbonium cations, oxonium cations and sulfonium cations and also cationic transition metal complexes. Particular mention may be made of the triphenylmethyl cation, the silver cation and the 1,1′-dimethylferrocenyl cation. They preferably have noncoordinating counterions, in particular boron compounds as are also mentioned in WO 91/09882, preferably tetrakis(pentafluorophenyl)borate.

Salts having noncoordinating anions can also be prepared by combining a boron or aluminum compound, e.g. an aluminum alkyl, with a second compound which can react to link two or more boron or aluminum atoms, e.g. water, and a third compound which, together with the boron or aluminum compound forms an ionizing ionic compound, e.g. triphenylchloromethane. In addition, a fourth compound which likewise reacts with the boron or aluminum compound, e.g. pentafluorophenol, can be added.

Ionic compounds containing Brönsted acids as cations preferably likewise have noncoordinating counterions. As Brönsted acid, particular preference is given to protonated amine or aniline derivatives. Preferred cations are N,N-dimethylanilinium, N,N-dimethylcylohexylammonium and N,N-dimethylbenzylammonium and also derivatives of the latter two.

Preferred ionic compounds as cocatalysts or cation-forming compounds are, in particular, N,N-dimethylanilinium tetrakis(pentafluorophenyl)borate, N,N-dimethylcyclohexylammonium tetrakis(pentafluorophenyl)borate or N,N-dimethylbenzylammonium tetrakis(pentafluorophenyl)borate.

It is also possible for two or more borate anions to be joined to one another, as in the dianion [(C₆F₅)₂B—C₆F₄—B(C₆F₅)₂]²⁻, or the borate anion can be bound via a bridge having a suitable functional group to the surface of a support particle.

Further suitable cocatalysts or cation-forming compounds are listed in WO 00/31090.

The amount of strong, uncharged Lewis acids, ionic compounds having Lewis-acid cations or ionic compounds containing Brönsted acids as cations is usually from 0.1 to 20 equivalents, preferably from 1 to 10 equivalents, based on the organometallic transition metal compound of the formula (I), in the process of the invention.

Suitable cocatalysts or cation-forming compounds also include boron-aluminum compounds such as di[bis(pentafluorophenyl)boroxy]methylalane. Examples of such boron-aluminum compounds are those disclosed in WO 99/06414.

It is also possible to use mixtures of all of the abovementioned cocatalysts or cation-forming compounds. Preferred mixtures comprise aluminoxanes, in particular methylaluminoxane, and an ionic compound, in particular one containing the tetrakis(pentafluorophenyl)borate anion, and/or a strong and uncharged Lewis acid, in particular tris(pentafluorophenyl)borane.

Both the organometallic transition metal compound of the formula (I) and the cocatalyst or cation-forming compounds are preferably used in a solvent, preferably an aromatic hydrocarbon having from 6 to 20 carbon atoms, in particular xylenes and toluene.

The catalyst in the process of the invention can further comprise a metal compound of the general formula (XV),

M⁴(R⁵²)ᵣ(R⁵³)ₛ(R⁵⁴)ₜ (XV)

where
M⁴ is an alkali metal, an alkaline earth metal or a metal group 13 of the Periodic Table, i.e. boron, aluminum, gallium, indium or thallium,
R⁵² is hydrogen, C₁-C₁₀-alkyl, C₈-C₁₅-aryl, alkylaryl or arylalkyl each having from 1 to 10 carbon atoms in the alkyl radical and from 6 to 20 carbon atoms in the aryl radical,
R⁵³ and R⁵⁴ are identical or different and are each hydrogen, halogen, C₁-C₁₀-alkyl, C₆-C₁₅-aryl, alkylaryl, arylalkyl or alkoxy each having from 1 to 10 carbon atoms in the alkyl radical and from 6 to 20 carbon atoms in the aryl radical,
r is an integer from 1 to 3,
   and
s and t are integers from 0 to 2, with the sum r+s+t corresponding to the valence of M⁴,
   where the metal compound of the formula (XV) is usually not identical to the cocatalyst or the cation-forming compound. It is also possible to use mixtures of various metal compounds of the formula (XV).

Among the metal compounds of the general formula (XV), preference is given to those in which
M⁴ is lithium, magnesium or aluminum and
R⁵³ and R⁵⁴ are each C₁-C₁₀-alkyl.

Particularly preferred metal compounds of the formula (XV) are n-butyllithium, n-butyl-n-octylmagnesium, n-butyl-n-heptylmagnesium, tri-n-hexylaluminum, triisobutylaluminum, triethylaluminum and trimethylaluminum and mixtures thereof.

When a metal compound of the formula (XV) is used, it is preferably present in the catalyst in such an amount that the molar ratio of M⁴ from formula (XV) to transition metal M¹ from the organometallic transition metal compound of the formula (I) is from 800:1 to 1:1, in particular from 200:1 to 2:1.

The catalyst system comprising an organometallic transition metal compound of the formula (I) or (Ia) and at least one cocatalyst can, depending on the polymerization process used, further comprise a support.

To obtain such a supported catalyst system, the unsupported catalyst system can be reacted with a support. The order in which support, the organometallic transition metal compound and the cocatalyst are combined is in principle immaterial. The organometallic transition metal compound and the cocatalyst can be immobilized independently of one another or simultaneously. After the individual process steps, the solid can be washed with suitable inert solvents, e.g. aliphatic or aromatic hydrocarbons.

As support, preference is given to using finely divided supports which can be any organic or inorganic, inert solid. In particular, the support can be a porous support such as talc, a sheet silicate, an inorganic oxide or a finely divided polymer powder (e.g. polyolefin).

Suitable inorganic oxides may be found among the oxides of elements of groups 2, 3, 4, 5, 13, 14, 15 and 16 of the Periodic Table of the Elements. Examples of oxides preferred as supports include silicon dioxide, aluminum oxide and mixed oxides of the elements calcium, aluminum, silicon, magnesium or titanium and also corresponding oxide mixtures. Other inorganic oxides which can be used alone or in combination with the abovementioned preferred oxidic supports are, for example, MgO, ZrO₂, TiO₂ or B₂O₃. A preferred mixed oxide is, for example, calcined hydrotalcite.

The support materials used preferably have a specific surface area in the range from 10 to 1000 m²/g, a pore volume in the range from 0.1 to 5 ml/g and a mean particle size of from 1 to 500 μm. Preference is given to supports having a specific surface area in the range from 50 to 500 m²/g, a pore volume in the range from 0.5 to 3.5 ml/g and a mean particle size in the range from 5 to 350 μm. Particular preference is given to supports having a specific surface area in the range from 200 to 400 m²/g, a pore volume in the range from 0.8 to 3.0 ml/g and a mean particle size of from 10 to 100 μm.

The inorganic supports can be subjected to a thermal treatment, e.g. to remove adsorbed water. Such a drying treatment is generally carried out at temperatures in the range from 80 to 300° C., preferably from 100 to 200° C., with drying at from 100 to 200° C. preferably being carried out under reduced pressure and/or under a blanket of inert gas (e.g. nitrogen), or the inorganic supports can be calcined at temperatures of from 200 to 1000° C. to produce the desired structure of the solid and/or to set the desired OH concentration on the surface. The support can also be treated chemically using customary desiccants such as metal alkyls, preferably aluminum alkyls, chlorosilanes or SiCl₄, or else methylaluminoxane. Appropriate treatment methods are described, for example, in WO 00/31090.

The inorganic support material can also be chemically modified. For example, treatment of silica gel with (NH₄)₂SiF₆ leads for fluorination of the silica gel surface, or treatment of silica gels with silanes containing nitrogen-, fluorine- or sulfur-containing groups leads to correspondingly modified silica gel surfaces.

Organic support materials such as finely divided polyolefin powders (e.g. polyethylene, polypropylene or polystyrene) can also be used and are preferably likewise freed of adhering moisture, solvent residues or other impurities by appropriate purification and drying operations before use. It is also possible to use functionalized polymer supports, e.g. ones based on polystyrene, via whose functional groups, for example ammonium or hydroxy groups, at least one of the catalyst components can be fixed.

In a preferred embodiment of the preparation of the supported catalyst system, at least one organometallic transition metal compound of the formula (I) or (Ia) is brought into contact with at least one cocatalyst as activating or cation-forming compound in a suitable solvent, giving a soluble or insoluble, preferably soluble, reaction product, an adduct or a mixture.

The preparation obtained in this way is then mixed with the dehydrated or passivated support material, the solvent is removed and the resulting supported organometallic transition metal compound catalyst system is dried to ensure that all or most of the solvent is removed from the pores of the support material. The supported catalyst is usually obtained as a free-flowing powder. Examples of the industrial implementation of the above process are described in WO 96/00243, WO 98/40419 or WO 00/05277.

A further preferred embodiment comprises firstly applying the cocatalyst or the cation-forming compound to the support component and subsequently bringing this supported cocatalyst or this cation-forming compound into contact with the organometallic transition metal compound.

Cocatalyst systems which are likewise of importance are therefore combinations obtained by combining the following components:
1st component: at least one defined boron or aluminum compound,
2nd component: at least one uncharged compound having at least one acidic hydrogen atom,
3rd component at least one support, preferably an inorganic oxidic support, and optionally, as 4th component, a base, preferably an organic nitrogen-containing base such as an amine, an aniline derivative or a nitrogen heterocycle.

The boron or aluminum compound used in the preparation of these supported cocatalysts are preferably compounds of the formula (XVI)

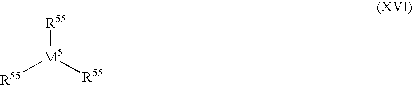

where
the radicals R⁵⁵ are identical or different and are each hydrogen, halogen, C₁-C₂₀-alkyl, C₁-C₂₀-haloalkyl, C₁-C₁₀-alkoxy, C₆-C₂₀-aryl, C₆-C₂₀-haloaryl, C₆-C₂₀-aryloxy, C₇-C₄₀-arylalkyl, C₇-C₄₀-haloarylalkyl, C₇-C₄₀-alkylaryl, C₇-C₄₀-haloalkylaryl, or R⁵⁵ is an OSiR⁵⁶₃ group, where
the radicals R⁵⁶ are identical or different and are each hydrogen, halogen, C₁-C₂₀-alkyl, C₁-C₂₀-haloalkyl, C₁-C₁₀-alkoxy, C₆-C₂₀-aryl, C₆-C₂₀-haloaryl, C₆-C₂₀-aryloxy, C₇-C₄₀-arylalkyl, C₇-C₄₀-haloarylalkyl, C₇-C₄₀-alkylaryl, C₇-C₄₀-haloalkylaryl, preferably hydrogen, C₁-C₈-alkyl or C₇-C₂₀-arylalkyl, and
M⁵ is boron or aluminum, preferably aluminum.

Particularly preferred compounds of the formula (XVI) are trimethylaluminum, triethylaluminum and triisobutylaluminum.

The uncharged compounds which have at least one acidic hydrogen atom and can react with compounds of the formula (XVI) are preferably compounds of the formulae (XVII), (XVIII) or (XIX),

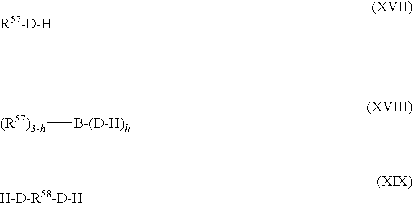

where
the radicals R⁵⁷ are identical or different and are each hydrogen, halogen, a boron-free organic radical having from 1 to 40 carbon atoms, e.g. C₁-C₂₀-alkyl, C₁-C₂₀-haloalkyl, C₁-C₁₀-alkoxy, C₆-C₂₀-aryl, C₆-C₂₀-haloaryl, C₆-C₂₀-aryloxy, C₇-C₄₀-arylalkyl, C₇-C₄₀-haloarylalkyl, C₇-C₄₀-alkylaryl, C₇-C₄₀-haloalkylaryl, an Si(R⁵⁹)₃ radical or a CH(SiR⁵⁹₃)₂ radical, where
R⁵⁹ is a boron-free organic radical having from 1 to 40 carbon atoms, e.g. C₁-C₂₀-alkyl, C₁-C₂₀-haloalkyl, C₁-C₁₀-alkoxy, C₆-C₂₀-aryl, C₆-C₂₀-haloaryl, C₆-C₂₀-aryloxy, C₇-C₄₀-arylalkyl, C₇-C₄₀-haloarylalkyl, C₇-C₄₀-alkylaryl, C₇-C₄₀-haloalkylaryl, and
R⁵⁸ is a divalent organic group having from 1 to 40 carbon atoms, e.g. C₁-C₂₀-alkylene, C₁-C₂₀-haloalkylene, C₆-C₂₀-arylene, C₆-C₂₀-haloarylene, C₇-C₄₀-arylalkylene, C₇-C₄₀-haloarylalkylene, C₇-C₄₀-alkylarylene, C₇-C₄₀-haloalkylarylene,
D is an element of group 16 of the Periodic Table of the Elements or an NR⁶⁰ group, where R⁶⁰ is hydrogen or a C₁-C₂₀-hydrocarbon radical such as C₁-C₂₀-alkyl or C₆-C₂₀-aryl, with preference being given to D being oxygen, and
h is 1 or 2.

Suitable compounds of the formula (XVII) are water, alcohols, phenol derivatives, thiophenol derivatives or aniline derivatives, with the halogenated and in particular the perfluorinated alcohols and phenols being of particular importance. Examples of particularly useful compounds are pentafluorophenol, 1,1-bis(pentafluorophenyl)methanol and 4-hydroxy-2,2′,3,3′,4′,5,5′,6,6′-nonafluoro-biphenyl.

Suitable compounds of the formula (XVIII) are boronic acids and borinic acids, in particular borinic acids having perfluorinated aryl radicals, for example (C₆F₅)₂BOH.

Suitable compounds of the formula (XIX) are dihydroxy compounds in which the divalent carbon-containing bridge is preferably halogenated and in particular perfluorinated. An example of such a compound is 4,4′-dihydroxy-2,2′,3,3′,5,5′,6,6′-octafluorobiphenyl hydrate.

Examples of combinations of compounds of the formula (XVI) with compounds of the formula (XVII) or (XIX) are trimethylaluminum/pentafluorophenol, trimethylaluminum/1-bis(pentafluorophenyl)methanol, trimethylaluminum/4-hydroxy-2,2′,3,3′,4′,5,5′,6,6′-nonafluoro-biphenyl, triethylaluminum/pentafluorophenol or triisobutylaluminum/pentafluorophenol or triethylaluminum/4,4′-dihydroxy-2,2′,3,3′,5,5′,6,6′-octafluorobiphenyl hydrate, with, for example, reaction products of the following type being able to be formed.

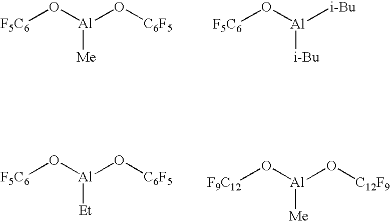

Examples of reaction products from the reaction of at least one compound of the formula (XVI) with at least one compound of the formula (XVIII) are:

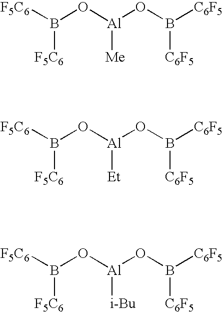

The order in which the components are combined is in principle immaterial.

The reaction products from the reaction of at least one compound of the formula (XVI) with at least one compound of the formula (XVII), (XVIII) or (XIX) and optionally the organic nitrogen base may, if appropriate, be additionally combined with an organometallic compound of the formula (XI), (XII), (XIII) and/or (XV) so as then to form, together with the support, the supported cocatalyst system.

In a preferred variant, the 1st component, e.g. compounds of the formula (XII), is combined with the 2nd component, e.g. compounds of the formula (XVII), (XVIII) or (XIX), and a support as 3rd component is combined with a base as 4th component and the two mixtures are subsequently reacted with one another, preferably in an inert solvent or suspension medium. The supported cocatalyst formed can be freed of the inert solvent or suspension medium before it is reacted with an organometallic transition metal compound of the formula (I) or (Ia) and, if appropriate, a metal compound of the formula (XV) to form the catalyst system.

It is also possible for the catalyst solid firstly to be prepolymerized with α-olefins, preferably linear C₂-C₁₀-1-alkenes and in particular ethylene or propylene, and the resulting prepolymerized catalyst solid then to be used in the actual polymerization. The mass ratio of catalyst solid used in the prepolymerization to monomer to be polymerized onto it is usually in the range from 1:0.1 to 1:200.

Furthermore, a small amount of an olefin, preferably an α-olefin, for example vinylcyclohexane, styrene or phenyldimethylvinylsilane, as modifying component, an antistatic or a suitable inert compound such as a wax or oil can be added as additive during or after the preparation of the supported catalyst system. The molar ratio of additives to organometallic transition metal compound according to the invention is usually from 1:1000 to 1000:1, preferably from 1:5 to 20:1.

The novel organometallic transition metal compounds of the formula (Ia) or the catalyst systems in which they are present are suitable for the polymerization or copolymerization of olefins, in particular for preparing polyolefin waxes.

In the process of the invention, the catalyst system is generally used together with a further metal compound of the general formula (XV), which may differ from the metal compound or compounds of the formula (XV) used in the preparation of the catalyst system, for the polymerization or copolymerization of olefins. The further metal compound is generally added to the monomer or the suspension medium and serves to free the monomer of substances which could adversely affect the catalyst activity. It is also possible to add one or more further cocatalytic or cation-forming compounds to the catalyst system in the polymerization process.

The invention is illustrated by following, nonrestrictive examples:

### EXAMPLES

### General

The synthesis and handling of the organometallic compounds and the catalysts was carried out in the absence of air and moisture under argon (glove box and Schlenk technique). All solvents used were purged with argon and dried over molecular sieves before use. Tetrahydrofuran (THF), diethyl ether and toluene were dried over sodium/benzophenone, pentane over sodium/benzophenone/triglyme and dichloromethane over calcium hydride by refluxing for a number of hours, subsequently distilled off and stored over 4 Å molecular sieves.

Methylaluminoxane (solution in toluene; 30% by weight of MAO) was procured from Albemarle Corp. and Al(iso-Bu)₃ (1 M solution In toluene) was procured from Aldrich Chemical Company. 2,5-Dimethyl-3-phenyl-6-H-cyclopenta[a]thiophene was synthesized as described in U.S. Pat. No. 6,444,833 and 2-methyl-1H-cyclopenta[a]naphthalene, also referred to as 2-methyl-4,5-benzoindene, was synthesized as described in U.S. Pat. No. 5,455,366.

Mass spectra were measured using a Hewlett Packard series 6890 instrument equipped with a series 5973 mass analyzer (EI, 70 eV).

NMR spectra of organic and organometallic compounds were recorded using a Varian Unity-300 NMR spectrometer at room temperature. The chemical shifts are reported relative to SiMe₄.

### Determination of the Melting Point:

The melting point Tₘ was determined by DSC in accordance with ISO standard 3146 in a first heating phase at a heating rate of 20° C. per minute up to 200° C., a dynamic crystallization at a cooling rate of 20° C. per minute down to 25° C. and a second heating phase at a heating rate of 20° C. per minute back to 200° C. The melting point was then the temperature at which the curve of enthalpy versus temperature measured in the second heating phase displayed a maximum.

### Gel Permeation Chromatography:

Gel permeation chromatography (GPC) was carried out at 145° C. in 1,2,4-trichlorobenzene using a 150 C GPC apparatus from Waters. The data were evaluated using the software Win-GPC from HS-Entwicklungsgesellschaft für wissenschaftliche Hard- und Software mbH, Ober-Hilbersheim. The calibration of the columns was carried out using polypropylene standards having molar masses of from 100 to 10⁷ g/mol. Mass average (M_{w}) and number average (Mₙ) molar masses of the polymers were determined. The Q value is the ratio of mass average molar mass (M_{w}) to number average molar mass (Mₙ).

### Determined of the Viscosity Number (I.V.):

The viscosity number was determined in decalin at 135° C. on an Ubbelohde viscosimeter PVS 1 using a measuring head S 5 (both from Lauda). To prepare the sample, 20 mg of polymer were dissolved in 20 ml of decalin at 135° C. for 2 hours. 15 ml of the solution were placed in the viscosimeter; the instrument carried out a minimum of three running-out time measurements until a consistent result had been obtained. The I.V. is calculated from the running-out times according to I.V.=(t/t₀−1)*1/cm where t=mean running-out time of the solution, t₀=mean running-out time of the solvent, c=concentration of the solution in g/ml.

### EXAMPLES

### 1. {Me₂Si(2,5-Me₂-3-Ph-cyclopenta[b]thiophen-6-yl)(2-Me-4,5-benzoindenyl)}ZrCl₂ (1)

### 1a) (2,5-Dimethyl-3-phenyl-6H-cyclopenta[b]thiophen-6-yl)dimethyl(2-methyl-3H-cyclopenta[a]naphthalen-3-yl)silane (1a)

A solution of 13 ml of n-butyllithium in hexane (2.5 M in hexane, 32.5 mmol) was slowly added at −78° C. to a solution of 6.4 g of 2,5-dimethyl-3-phenyl-6H-cyclopenta[b]thiophene (28.3 mmol) in 60 ml of diethyl ether and the mixture was subsequently stirred at room temperature for 16 hours. The solution was cooled to −78° C., 4.0 ml of dichlorodimethylsilane (33 mmol) were added and the reaction mixture was subsequently stirred at room temperature for 6 hours.

The precipitate was filtered off and the filtercake was washed with 30 ml of n-pentane. Solvent and excess dichlorodimethylsilane were removed under reduced pressure, and the residue was subsequently dissolved in 60 ml of tetrahydrofuran (THF).

In a second reaction vessel, 5.0 g of 2-methyl-1H-cyclopenta[α]naphthalene (28.3 mmol) were dissolved in 50 ml of diethyl ether, whose solution was cooled to −78° C. and admixed with a solution of 12 ml of n-butyllithium in hexane (2.5 M in hexane, 30 mmol). The reaction solution was stirred at room temperature for 12 hours, 0.24 ml of N-methylimidazole were added to the reddish solution and this solution was stirred for a further 15 minutes. This second solution was slowly added to the first solution of the chlorosilane prepared above in THF which had been cooled to 0° C. After stirring the reaction mixture at room temperature for 18 hours, 10 ml of an aqueous saturated ammonium chloride solution were added dropwise. The organic phase was separated off, diluted with 100 ml of diethyl ether, washed with a saturated aqueous solution of sodium chloride and dried over magnesium sulfate. The solvents were removed on a rotary evaporator and the residue was chromatographed on silica gel (eluent: 10% of methylene chloride in hexane). This gave 4.7 g (36% yield) of the ligand (1a).

EIMS: m/e (%) 462 (M+, 30), 283 (100), 255 (10), 237 (50), 221 (15), 195 (22), 178 (88), 152 (14).

### 1 {Me₂Si(2,5-Me₂-3-Ph-cyclopenta[b]thiophen-6-yl)(2-Me-4,5-benzoindenyl)}ZrCl₂ (1)

8.9 ml of a solution of n-butyllithium in hexane (2.5 M in hexane, 22.2 mmol) were added at 0° C. to a solution of 4.89 g of (2,5-dimethyl-3-phenyl-6H-cyclopenta[b]thiophen-6-yl)dimethyl(2-methyl-3H-cyclopenta[a]naphthalen-3-yl)silane (10.6 mmol) in 60 ml of diethyl ether. After stirring at room temperature for 16 hours, the solvents were removed under reduced pressure and 2.47 g of zirconium tetrachloride (10.6 mmol) were added to the dry powder. The mixture was stirred in 80 ml of a solvent mixture of hexane and diethyl ether (5/1 volume ratio) for 16 hours. The yellow precipitate formed was isolated with the aid of a glass filter frit, the filtercake was washed with pentane and dried under reduced pressure. The crude product (5.1 g) was stirred in 100 ml of methylene chloride and the suspension was filtered through Celite. The filtrate was evaporated by removing the solvent under reduced pressure. This gave 3.8 g of product (62%) (1) as a rac/meso mixture (1/1). 2 g of the rac/meso mixture was suspended in the presence of 150 mg of lithium chloride in THF and refluxed for 5 hours. After cooling, filtration through a glass filter frit, washing with pentane and drying under reduced pressure, 750 mg of the rac isomer were isolated as a yellow powder.

¹H-NMR (CDCl₃): (rac isomer) 8.1 (d, 1H), 7.8 (d, 1H), 7.6 (t, 1H), 7.5 (m, 2H), 7.4 (m, 3H), 7.32 (m, 3H), 7.23 (m, 1H), 6.4 (s, 1H), 2.52 (s, 3H), 2.48 (s, 3H), 2.17 (s, 3H), 1.3 (s, 3H), 1.15 (s, 3H).

### POLYMERIZATION EXAMPLES

### Example P1 Homopolymerization of Propene

4 ml of a solution of triisobutylaluminum in toluene (4 mmol, 1M) were placed in a dry 1 l reactor which had been flushed with nitrogen. 250 g of propylene were introduced at 30° C. and the contents of the reactor were heated to a temperature of 65° C. A catalyst solution obtained by combining 0.4 ml of a solution of metallocene (1) from Example 1 in toluene, which had been prepared from 1.3 mg of metallocene (1) and 10 ml of toluene, with 0.8 ml of a solution of methylaluminoxane in toluene (3.8 mmol, 30% by weight) and subsequently allowing the mixture to react further for 10 minutes was introduced into the reactor together with 50 g of propylene. The contents of the reactor was stirred at 65° C. for 0.25 hour and the polymerization reaction was stopped by venting the reactor. On evaporation of the propene, the reactor cooled down to room temperature. After flushing the reactor with nitrogen for 10 minutes, 5 ml of methanol were added to the contents of the reactor. The polymer was taken out and dried at 50° C. under reduced pressure in a vacuum drying oven for one hour. 21.4 g of polypropylene were obtained. The results of the polymerization and the results of the polymer analysis are shown in Table 1 below.

### Example P2 Homopolymerization of Propene

The polymerization was carried out in manner analogous to Example P1. 4 ml of a solution of triisobutylaluminum in toluene (4 mmol, 1M) were placed in the reactor together with 250 g of propylene at 30° C., the mixture was heated to 65° C. and 0.1 standard liter of hydrogen (4.1 mmol) were fed in. A catalyst solution obtained by combining 0.3 ml of a solution of metallocene (1) from Example 1 in toluene, which had been prepared from 1.0 mg of metallocene (1) and 10 ml of toluene, with 0.8 ml of a solution of methylaluminoxane in toluene (3.8 mmol, 30% by weight) and subsequently allowing the mixture to react further for 10 minutes was introduced into the reactor together with 50 g of propylene. The contents of the reactor was stirred at 65° C. for 0.25 hour. After stopping the reaction and working up the polymer, 54.4 g of polypropylene were obtained. The results of the polymerization and the results of the polymer analysis are shown in Table 1 below.

### Example P3 Homopolymerization of Propene

The polymerization was carried out in manner analogous to Example P2. 4 ml of a solution of triisobutylaluminum in toluene (4 mmol, 1M) were placed in the reactor together with 250 g of propylene at 30° C., the mixture was heated to 65° C. and 0.3 standard liter of hydrogen (12.3 mmol) were fed in. A catalyst solution obtained by combining 0.2 ml of a solution of metallocene (1) from Example 1 in toluene, which had been prepared from 1.7 mg of metallocene (1) and 10 ml of toluene, with 0.8 ml of a solution of methylaluminoxane in toluene (3.8 mmol, 30% by weight) and subsequently allowing the mixture to react further for 10 minutes was introduced into the reactor together with 50 g of propylene. The contents of the reactor was stirred at 65° C. for 0.25 hour. After stopping the reaction and working up the polymer, 69.8 g of polypropylene were obtained. The results of the polymerization and the results of the polymer analysis are shown in Table 1 below.

In the following comparative examples, the following two metallocenes were used in place of the metallocene (1) from Example 1:
A: {Me₂Si(2-Me-4,5-benzoindenyl)₂}ZrCl₂ (A)
B: {Me₂Si(2,5-Me₂-3-Ph-cyclopenta[b]thiophen-6-yl)₂}ZrCl₂ (B)

### Example CP1 Homopolymerization of Propene

The polymerization was carried out in manner analogous to Example P1. 4 ml of a solution of triisobutylaluminum in toluene (4 mmol, 1M) were placed in the reactor together with 250 g of propylene at 30° C. and the mixture was heated to 65° C. A catalyst solution obtained by combining 0.7 ml of a solution of metallocene (A) in toluene, which had been prepared from 2.0 mg of metallocene (A) and 10 ml of toluene, with 0.8 ml of a solution of methylaluminoxane in toluene (3.8 mmol, 30% by weight) and subsequently allowing the mixture to react further for 10 minutes was introduced into the reactor together with 50 g of propylene. The contents of the reactor was stirred at 65° C. for 0.25 hour. After stopping the reaction and working up the polymer, 31.3 g of polypropylene were obtained. The results of the polymerization and the results of the polymer analysis are shown in Table 1 below.

### Example CP2 Homopolymerization of Propene

The polymerization was carried out in manner analogous to Example P2. 4 ml of a solution of triisobutylaluminum in toluene (4 mmol, 1M) were placed in the reactor together with 250 g of propylene at 30° C., the mixture was heated to 65° C. and 0.1 standard liter of hydrogen (4.1 mmol) were fed in. A catalyst solution obtained by combining 1.2 ml of a solution of metallocene (A) in toluene, which had been prepared from 1.4 mg of metallocene (A) and 10 ml of toluene, with 0.8 ml of absolution of methylaluminoxane in toluene (3.8 mmol, 30% by weight) and subsequently allowing the mixture to react further for 10 minutes was introduced into the reactor together with 50 g of propylene. The contents of the reactor was stirred at 65° C. for 0.25 hour. After stopping the reaction and working up the polymer, 61.3 g of polypropylene were obtained. The results of the polymerization and the results of the polymer analysis are shown in Table 1 below.

### Example CP3 Homopolymerization of Propene

The polymerization was carried out in manner analogous to Example P3. 4 ml of a solution of triisobutylaluminum in toluene (4 mmol, 1 M) were placed in the reactor together with 250 g of propylene at 30° C., the mixture was heated to 65° C. and 0.4 standard liter of hydrogen (16.4 mmol) were fed in. A catalyst solution obtained by combining 1.2 ml of a solution of metallocene (A) in toluene, which had been prepared from 1.4 mg of metallocene (A) and 10 ml of toluene, with 0.8 ml of a solution of methylaluminoxane in toluene (3.8 mmol, 30% by weight) and subsequently allowing the mixture to react further for 10 minutes was introduced into the reactor together with 50 g of propylene. The contents of the reactor was stirred at 65° C. for 0.25 hour. After stopping the reaction and working up the polymer, 43.7 g of polypropylene were obtained. The results of the polymerization and the results of the polymer analysis are shown in Table 1 below.

### Example CP4 Homopolymerization of Propene

The polymerization was carried out in manner analogous to Example CP1. 4 ml of a solution of triisobutylaluminum in toluene (4 mmol, 1M) were placed in the reactor together with 250 g of propylene at 30° C. and the mixture was heated to 65° C. A catalyst solution obtained by combining 0.7 ml of a solution of metallocene (B) in toluene, which had been prepared from 1.4 mg of metallocene (B) and 10 ml of toluene, with 0.8 ml of a solution of methylaluminoxane in toluene (3.8 mmol, 30% by weight) and subsequently allowing the mixture to react further for 10 minutes was introduced into the reactor together with 50 g of propylene. The contents of the reactor was stirred at 65° C. for 0.25 hour. After stopping the reaction and working up the polymer, 47.2 g of polypropylene were obtained. The results of the polymerization and the results of the polymer analysis are shown in Table 1 below.

### Example CP5 Homopolymerization of Propene

The polymerization was carried out in manner analogous to Example CP2. 4 ml of a solution of triisobutylaluminum in toluene (4 mmol, IM) were placed in the reactor together with 250 g of propylene at 30° C., the mixture was heated to 65° C. and 0.1 standard liter of hydrogen (4.1 mmol) were fed in. A catalyst solution obtained by combining 0.7 ml of a solution of metallocene (B) in toluene, which had been prepared from 1.4 mg of metallocene (B) and 10 ml of toluene, with 0.8 ml of a solution of methylaluminoxane in toluene (3.8 mmol, 30% by weight) and subsequently allowing the mixture to react further for 10 minutes was introduced into the reactor together with 50 g of propylene. The contents of the reactor was stirred at 65° C. for 0.25 hour. After stopping the reaction and working up the polymer, 52.2 g of polypropylene were obtained. The results of the polymerization and the results of the polymer analysis are shown in Table 1 below.

### Example CP6 Homopolymerization of Propene

The polymerization was carried out in manner analogous to Example CP3. 4 ml of a solution of triisobutylaluminum in toluene (4 mmol, 1 M) were placed in the reactor together with 250 g of propylene at 30° C., the mixture was heated to 65° C. and 0.4 standard liter of hydrogen (16.4 mmol) were fed in. A catalyst solution obtained by combining 0.5 ml of a solution of metallocene (B) in toluene, which had been prepared from 2.0 mg of metallocene (B) and 10 ml of toluene, with 0.8 ml of a solution of methylaluminoxane in toluene (3.8 mmol, 30% by weight) and subsequently allowing the mixture to react further for 10 minutes was introduced into the reactor together with 50 g of propylene. The contents of the reactor was stirred at 65° C. for 0.25 hour. After stopping the reaction and working up the polymer, 66.7 g of polypropylene were obtained. The results of the polymerization and the results of the polymer analysis are shown in Table 1 below.

TABLE 1

| | Metal- | Amount of | Activity | Viscosity | | |
|---|---|---|---|---|---|---|
| Exam- | locene | hydrogen | [kg/(mmol | number | M_{w} | Tₘ |
| ple | [No.] | [mmol] | * h)] | [dl/g] | [kg/mol] | [° C.] |
| P1 | (1) | 0 | 1615 | 3.26 | 496 | 151 |
| P2 | (1) | 4.1 | 4353 | 1.18 | 134 | 153 |
| P3 | (1) | 12.3 | 5798 | 0.56 | 51 | 151 |
| CP1 | (A) | 0 | 538 | 2.52 | 356 | 149 |
| CP2 | (A) | 4.1 | 1415 | 1.52 | 185 | 151 |
| CP3 | (A) | 16.4 | 1009 | 0.98 | 105 | 153 |
| CP4 | (B) | 0 | 1258 | 3.80 | 604 | 160 |
| CP5 | (B) | 4.1 | 1397 | 2.47 | 347 | 157 |
| CP6 | (B) | 16.4 | 1784 | 0.99 | 107 | 160 |

Units and abbreviations: Activity in kg_{polymer}/(mmol_{(transition metal compound} * h_{polymerization time}); weight average molar mass determined by GPC; polydispersity Q = Mₙ/M_{w}.

1. A process for preparing olefin polymers having a molar mass M_{w} of from 500 to 50 000 g/mol by polymerization or copolymerization of at least one olefin of the formula R^{a}—CH═CH—R^{b}, where R^{a} and R^{b} are identical or different and are each a hydrogen atom or a hydrocarbon radical having from 1 to 20 carbon atoms, or R^{a} and R^{b} together with the atoms connecting them can form a ring, at a temperature of from −60 to 200° C. and a pressure of from 0.5 to 100 bar, in solution, in suspension or in the gas phase, in the presence of hydrogen and in the presence of a catalyst system comprising at least one organometallic transition metal compound and at least one cocatalyst, wherein the organometallic transition metal compound is a compound of the formula (I), 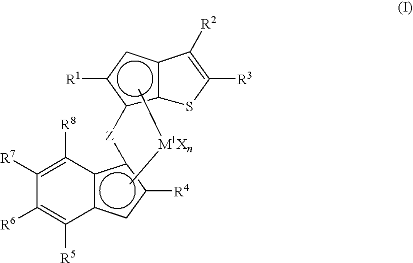
where
M¹ is an element of group 3, 4, 5 or 6 of the Periodic Table of the Elements or the lanthanides,
the radicals X are identical or different and are each an organic or inorganic radical, with two radicals X also being able to be joined to one another to form a divalent radical,
n is a natural number from 1 to 4,
R¹ is hydrogen, an organic radical which has from 1 to 40 carbon atoms and is unbranched in the α position or an organic radical which has from 1 to 40 carbon atoms and is bound via an sp²-hybridized carbon atom,
R² is an organic radical having from 1 to 40 carbon atoms,
R³ is an organic radical having from 1 to 40 carbon atoms,
R⁴ is hydrogen, an organic radical which has from 1 to 40 carbon atoms and is unbranched in the α position or an organic radical which has from 1 to 40 carbon atoms and is bound via an sp²-hybridized carbon atom,
R⁵, R⁶, R⁷, R⁸ are identical or different and are each hydrogen, halogen or an organic radical having from 1 to 40 carbon atoms or two adjacent radicals R⁵, R⁶, R⁷ or R⁸ together with the atoms connecting them form a monocyclic or polycyclic, substituted or unsubstituted ring system which has from 1 to 40 carbon atoms and may also contain heteroatoms selected from the group consisting of the elements Si, Ge, N, P, O, S, Se and Te,
and
Z is a bridge consisting of a divalent atom or a divalent group.

2. The process according to claim 1, wherein, in formula (I),
R¹ is a C₁-C₁₀-n-alkyl radical,
R² is a substituted or unsubstituted C₆-C₄₀-aryl radical or C₂-C₄₀-heteroaromatic radical having at least one heteroatom selected from the group consisting of the elements O, N, S and P,
R³ is a C₁-C₁₀-n-alkyl radical or a substituted or unsubstituted C₆-C₄₀-aryl radical or C₂-C₄₀-heteroaromatic radical having at least one heteroatom selected from the group consisting of the elements O, N, S and P,
and
the other variables and indices are as defined for formula (I).

3. The process according to claim 1 or 2, wherein, in formula (I),
R⁴ is a C₁-C₁₀-n-alkyl radical,
R⁵, R⁶ together form a substituted or unsubstituted 1,3-butadien-1,4-diyl group,
R⁷, R⁸ are each hydrogen,
and
the other variables and indices are as defined for formula (I).

4. The process according to any of claims 1 to 3, wherein propene is used as olefin.

5. The process according to any of claims 1 to 4, wherein an aluminoxane is used as cocatalyst.

6. The process according to claim 5, wherein the organometallic transition metal compound of the formula (I) is preactivated by means of an aluminoxane prior to use in the polymerization reaction.

7. An organometallic transition metal compound of the formula (Ia) 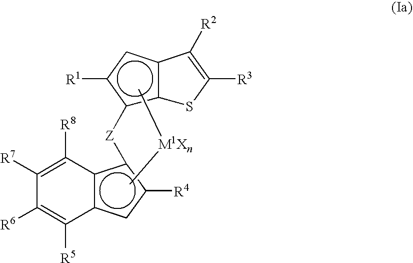
where
M¹ is an element of group 3, 4, 5 or 6 of the Periodic Table of the Elements or the lanthanides,
the radicals X are identical or different and are each an organic or inorganic radical, with two radicals X also being able to be joined to one another to form a divalent radical,
n is a natural number from 1 to 4,
R¹ is hydrogen, an organic radical which has from 1 to 40 carbon atoms and is unbranched in the α position or an organic radical which has from 1 to 40 carbon atoms and is bound via an sp²-hybridized carbon atom,
R² is an organic radical having from 1 to 40 carbon atoms,
R³ is an organic radical having from 1 to 40 carbon atoms,
R⁴ is hydrogen, an organic radical which has from 1 to 40 carbon atoms and is unbranched in the α position or an organic radical which has from 1 to 40 carbon atoms and is bound via an sp²-hybridized carbon atom,
R⁵, R⁶ together form a substituted or unsubstituted 1,3-butadien-1,4-diyl group,
R⁷, R⁸ are identical or different and are each hydrogen, halogen or an organic radical having from 1 to 40 carbon atoms,
and
Z is a bridge consisting of a divalent atom or a divalent group.

8. A biscyclopentadienyl ligand system of the formula (II) 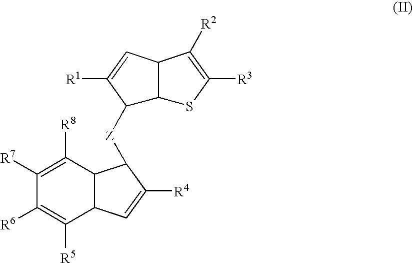
or its double bond isomers,
where the variables R¹, R², R³, R⁴, R⁵, R⁶, R⁷, R⁸ and Z are as defined for formula (Ia).

9. A catalyst system comprising at least one organometallic transition metal compound according to claim 7 and at least one cocatalyst.

10. The use of a biscyclopentadienyl ligand system according to claim 8 for preparing an organometallic transition metal compound.
